(19) United States
(12) Patent Application Publication
(10) Pub. No.: US 2020/0300768 A1
(43) Pub. Date: Sep. 24, 2020

(54) DETERMINATION DEVICE, DETERMINATION METHOD, AND DETERMINATION PROGRAM

(71) Applicant: NIKON CORPORATION, Tokyo (JP)

(72) Inventors: Muneaki HIKITA, Tokyo (JP); Makiko TAKUBO, Sagamihara-shi (JP)

(73) Assignee: NIKON CORPORATION, Minato-ku, Tokyo (JP)

(21) Appl. No.: 16/843,103

(22) Filed: Apr. 8, 2020

Related U.S. Application Data
(63) Continuation of application No. PCT/JP2018/028753, filed on Jul. 31, 2018.
(60) Provisional application No. 62/570,716, filed on Oct. 11, 2017.

(51) Int. Cl. G01N 21/65; G01N 33/483; G06N 20/00; G06N 5/04

(52) U.S. Cl. CPC G01N 21/65; G01N 33/4833; G01N 2201/126; G06N 5/04; G06N 20/00

(57) ABSTRACT

A determination device is provided which includes a determination unit which refers to a learned model generated by learning a teacher data including an optical spectrum measured from a cancer tissue whose primary focus is known, and determines a primary focus of a biological specimen according to input data based on an optical spectrum of a biological specimen measured by a measurement unit.

The contents of the following U.S. provisional application and international application are incorporated herein by reference:

No. 62/570,716 filed on Oct. 11, 2017; and

No. PCT/JP2018/028753 filed on Jul. 31, 2018

### BACKGROUND

### 1. Technical Field

The present invention relates to a determination device, a determination method, and a determination program.

### 2. Related Art

There is a demand for identifying a primary focus from a specimen of metastatic cancer, and a method is proposed to determine, from the specimen, that the specimen is derived from a stomach cancer (for example, see Patent document 1).

Patent document 1 Japanese Patent Application Publication No. 2004-321102

The known determination method is a method to check whether or not a specimen is derived from a stomach cancer, and cannot identify a primary focus of the specimen from a plurality of candidates of a primary focus.

### SUMMARY

In the first aspect of the present invention, a determination device is provided including: a determination unit which refers to a learned model generated by learning teacher data including an optical spectrum measured from the cancer tissue whose primary focus is known, and determines a primary focus of the biological specimen according to input data based on the optical spectrum of an unknown biological specimen.

In the second aspect of the present invention, a determination method is provided including a step of referring to the learned model generated by learning the teacher data including the optical spectrum measured from the cancer tissue whose primary focus is known and determining the primary focus of the biological specimen according to input data based on the optical spectrum measured from an unknown biological specimen.

In the third aspect of the present invention, a determination program is provided to enable a computer to perform a step of referring a learned model generated by learning the teacher data including the optical spectrum measured from the cancer tissue whose primary focus is known, and determining the primary focus of the biological specimen based on input data corresponding to the optical spectrum measured from the unknown biological specimen.

The summary clause does not necessarily describe all necessary features of the embodiments of the present invention. The present invention may also be a sub-combination of the features described above.

### BRIEF DESCRIPTION OF THE DRAWINGS

FIG. 1 is a schematic view showing a structure of the determination device 100.

FIG. 2 is a flow diagram showing a procedure to generate a learned model.

FIG. 3 is a schematic view showing the region of interest 310.

FIG. 4 is an exemplary diagram showing a model of neural network.

FIG. 5 is a flow diagram showing a procedure to determine a biological specimen.

FIG. 6 is a schematic view showing a structure of the determination unit 210.

FIG. 7 is an exemplary diagram showing an optical spectrum measured from a biological specimen.

FIG. 8 is a diagram showing a state in which dimensions are reduced in a case in which a measurement optical spectrum is learned.

FIG. 9 is a graph showing an integrated value of light intensities of intrinsic fluorescence.

FIG. 10 is a graph showing an integrated value of optical spectrums measured from a biological specimen.

FIG. 11 is a graph showing an integrated value of light intensities of intrinsic fluorescence for another biological specimen.

FIG. 12 is a schematic view showing the region of interest 310.

FIG. 13 is a diagram showing a process of clustering of optical spectrums.

FIG. 14 is a histogram showing a result of clustering of optical spectrums.

### DESCRIPTION OF EXEMPLARY EMBODIMENTS

Hereinafter, some embodiments of the present invention will be described. The following embodiments do not limit the invention according to the claims. In addition, not all combinations of features described in the embodiments are essential for the solution of the invention.

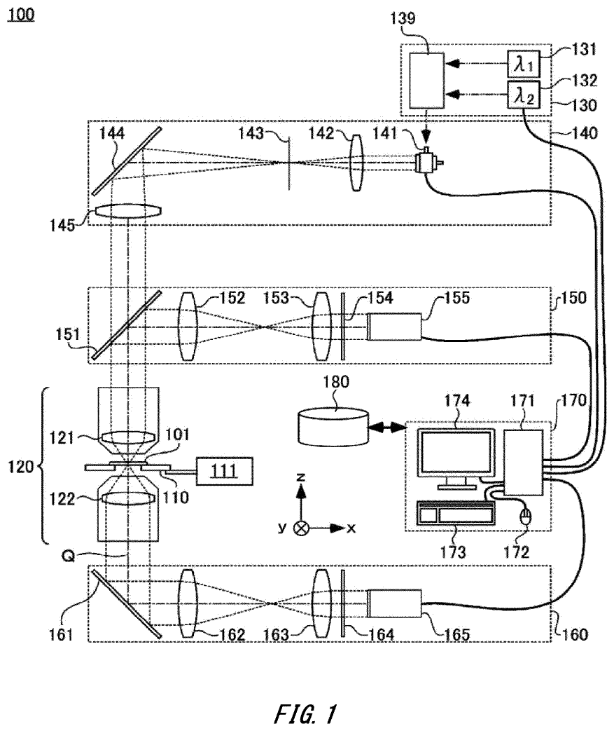

FIG. 1 is a schematic view showing a structure of the determination device 100. The determination device 100 includes the stage 110, the objective optical system 120, the light source device 130, the irradiation optical system 140, the front detection unit 150, the rear detection unit 160, the control unit 170, and the storage unit 180. It is noted that in the following description, the “optical spectrum” is simply described as “spectrum”.

In the determination device 100, the stage 110, the objective optical system 120, the light source device 130, the irradiation optical system 140, the front detection unit 150, and the rear detection unit 160 constitute the measurement unit to measure a spectrum of the sample 101. The storage unit 180 stores a learned model described below. The processor 171 of the control unit 170 constitutes a determination unit to determine a primary focus of a cancer tissue together with the storage unit 180. The processor 171 also performs machine-learning to generate a learned model.

It is noted that, in the illustrated example, the storage unit 180 is provided as a part of the determination device 100. However, as long as the processor 171 can refer to the learned model stored in the storage unit 180, the storage unit 180 may be, for example, an online storage or a cloud storage disposed on other locations through a communication line.

The stage 110 of the determination device 100 supports the sample 101 which is the target for the determination by the determination device 100. The sample 101 includes a container or support, and a biological specimen. The container or support in the sample 101 is a container, a board, and the like formed of a material, such as glass, which is transparent for excitation light and Raman scattered light. The biological specimen means a sample which is a small piece including organs, tissues, or cells collected from a human or animal.

The stage 110 supports the container at the periphery. The stage 110 also has an opening in a part which does not support the container, and also exposes the container to the stage 110 side. Thereby, the sample 101 placed on the stage 110 can be irradiated with an excitation light from the stage 110 side, and the scattered light produced from the sample 101 can be observed from the stage 110 side.

The stage 110 also has the stage scanner 111. The stage scanner 111 moves the sample 101 in the x-y direction parallel to the surface on which the sample 101 is placed, and the z direction perpendicular thereto, as indicated with arrow x-y-z in the diagram. Thereby, in the determination device 100, a three-dimensional region in the sample 101 can be a target region for observation or determination while the optical axis of the optical system and the optical path of excitation light are fixed. It is noted that, in the following description, the region in the sample 101 which is the target for observation or determination by the determination device 100 is described as a region of interest.

The objective optical system 120 has the front objective lens 121 and the rear objective lens 122 arranged on the opposite sides to each other with respect to the stage 110. The front objective lens 121 serves to collect lights such as excitation light and illumination light with which the sample 101 is irradiated.

The light source device 130 has a plurality of the light source 131 and 132, and the combiner 139. The light source 131 and 132 produce irradiation light different from each other. The combiner 139 combines the lights produced by the light source 131 and 132. Thus, the irradiation light emitted from the light source 131 and 132 becomes a beam passing through a single optical path due to the combiner 139, and the sample 101 is irradiated with the beam at the same position.

The light source 131 produces an excitation light to be used in a case where the Raman spectroscopy of the sample 101 is measured, for example, a laser beam with a wavelength of 532 nm. The light source 132 may also produce an illumination light in a visible light band used in a case where the microscopic image of the sample 101 is observed. Furthermore, Raman scattered light may be produced with CARS process by using the light source 131 and 132 as a light source for pump ray and stokes ray. It is noted that the irradiation light with which the sample 101 is irradiated has preferably long wavelength which is unlikely to invade a living cell regardless of whether the irradiation light is excitation light or illumination light.

The irradiation optical system 140 has the galvano scanner 141 and the scan lens 142. The galvano scanner 141 includes a pair of reflection mirrors to swing around two swing axes which are not parallel to each other. Thereby, the optical path of the light entering the galvano scanner 141 two-dimensionally displaces in the direction crossing the optical axis.

The scan lens 142 focuses the excitation light emitted from the galvano scanner 141 on the predetermined primary image plane 143. Furthermore, the excitation light collimated by the collimator lens 145 arranged in the opposite side of the reflection mirror 144 which bend the optical path of excitation light is collected at the sample 101 by the front objective lens 121. Thus, any region of interest which is set in the sample 101 is irradiated with the excitation light emitted from the light source device 130.

The front detection unit 150 has the dichroic mirror 151, the relay lens 152 and 153, the band pass filter 154, and the spectrometer 155. The dichroic mirror 151 transmits the excitation light with which the sample 101 is irradiated from the collimator lens 145 with high efficiency. The dichroic mirror 151 also reflects the scattered light produced by the sample 101 with high efficiency.

The dichroic mirror 151 reflects the Raman scattered light produced in the sample 101 irradiated with the excitation light and directs it to the relay lens 152 and 153. The band pass filter 154 transmits the Raman scattered light produced by the sample 101 and allows it to enter the spectrometer 155, while absorbing or reflecting excitation light and Rayleigh scattered light. Thereby, the spectrometer 155 efficiently detects the Raman scattered light produced by the sample 101 in the direction of reflection and outputs the spectral image.

A polychromator and the like can be used as the spectrometer 155. It is noted that in the front detection unit 150, by placing an image sensor instead of the spectrometer 155, the determination device 100 can be used as a microscope.

The rear detection unit 160 has the reflection mirror 161, the relay lens 162 and 163, the band pass filter 164, and the spectrometer 165. The reflection mirror 161 reflects the Raman scattered light produced by the sample 101 and directs it to the relay lens 162 and 163, the band pass filter 164, and the spectrometer 165. It is noted that a dichroic mirror to selectively reflect a wavelength of the Raman scattered light may be provided instead of the reflection mirror 161.

The band pass filter 164 transmits the Raman scattered light produced by the sample 101 and allows it to enter the spectrometer 165, while absorbing or reflecting Rayleigh scattered light and excitation light. Thereby, the spectrometer 165 efficiently detects the Raman spectroscopy due to the transmitted light from the sample 101.

A polychromator and the like can be used as the spectrometer 165. It is noted that, in the rear detection unit 160, the determination device 100 can be used as a microscope by arranging an image sensor to detect a light in a visible light band, instead of the spectrometer 165.

In the determination device 100, a Raman scattered light detected by the front detection unit 150 disposed on the same side as the irradiation optical system 140 with respect to the sample 101 is a backward Raman scattered light reflected by the sample 101. On the other hand, a Raman scattered light detected by the rear detection unit 160 disposed on the opposite side to the irradiation optical system 140 with respect to the sample 101 is a forward Raman scattered light just like transmitted from the sample 101.

The control unit 170 includes the processor 171, the mouse 172, the keyboard 173, and the display unit 174. The mouse 172 and the keyboard 173 are connected to the processor 171, and operated when an instruction from the user is input to the processor 171.

The display unit 174 returns a feedback for the user operation of the mouse 172 and the keyboard 173, and also displays, for the user, an image or character string generated by the processor 171. Furthermore, in the determination device 100, the display unit 174 displays an observation image in which the sample 101 is optically observed, and characters, images or the like representing the determination result.

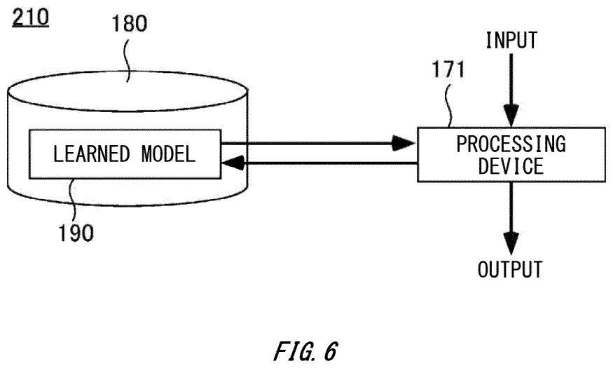

The storage unit 180 stores the learned model 190 (see FIG. 6) to which the determination device 100 refers when performing the determination operation. The learned model 190 may be the learned model 190 generated by means of the machine-learning of the determination device 100 itself, or may be the learned model 190 acquired through a communication line or a storage medium.

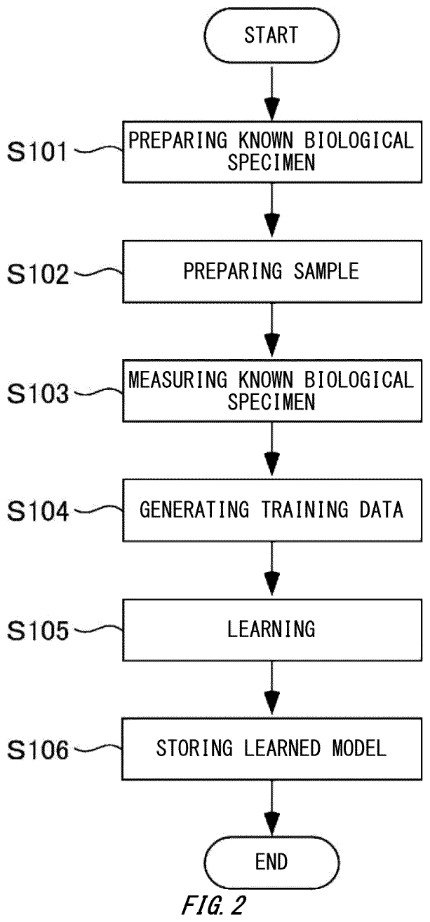

FIG. 2 is a flow diagram showing a procedure to create a learned model stored in the storage unit 180 of the determination device 100. When the learned model is created, a biological specimen is first prepared whose attribute to be determined is known (Step S101).

As an example of a biological specimen whose attribute to be determined is known, a known cancer tissue is used. Examples of a known cancer tissue include a metastatic cancer tissue whose primary focus is known, and a tissue known to include a cancer tissue. That is, when an attribute to be determined by the determination device 100 is a type of a primary focus of a biological specimen of an unknown metastatic cancer, a biological specimen of a cancer tissue whose primary focus is known is prepared. In addition, when an attribute to be determined by the determination device 100 is whether or not the cancer tissue exists, a biological specimen known to include a cancer tissue and a biological specimen known not to include the cancer tissue are prepared.

Then, the sample 101 of the biological specimen prepared in Step S101 is prepared so that the spectrum can be measured by the determination device 100 (Step S102). The sample 101 can be prepared using well-known various methods to prepare a biological specimen for a histopathologic examination.

For example, a biopsy tissue collected with an endoscope and the like is fixed with formalin, embedded with paraffin, and then sliced with a microtome. Furthermore, the obtained slice is placed on a slide glass (support), deparaffinized with xylene, and then dried, completing the sample 101. After deparaffinization, a cover glass may be placed to make a preparation specimen.

Then, all spectrums are measured for each of the prepared sample 101 using the determination device 100 (Step S103). That is, in the determination device 100, the sample 101 placed on the stage 110 is irradiated with excitation light, and, from the generated Raman scattered light, the all spectrums are measured using at least one of the spectrometer 155 and 165. Thus, an optical spectrum can be measured from a known cancer tissue.

Herein, the all spectrums means spectrums corresponding to the entire region of the regions of interest in the sample 101. The region of interest herein is a region which is the target for the measurement for a Raman spectrum from the biological specimen for the purpose of determination, and is set by a user of the determination device 100.

When a target for determination by the determination device 100 is a cancer tissue of a biological specimen, the biological specimen needs to be identified by the level of the cell size. Therefore, the regions of interest which is the target for the determination preferably contains a size equal to the size of a cancer cell, for example, equal to or larger than 5 μm² and equal to or less than 30 μm², or equal to or larger than 10 μm² and equal to or less than 20 μm². In addition, the regions of interest are set in a region on a biological specimen which is likely to include a cancer tissue.

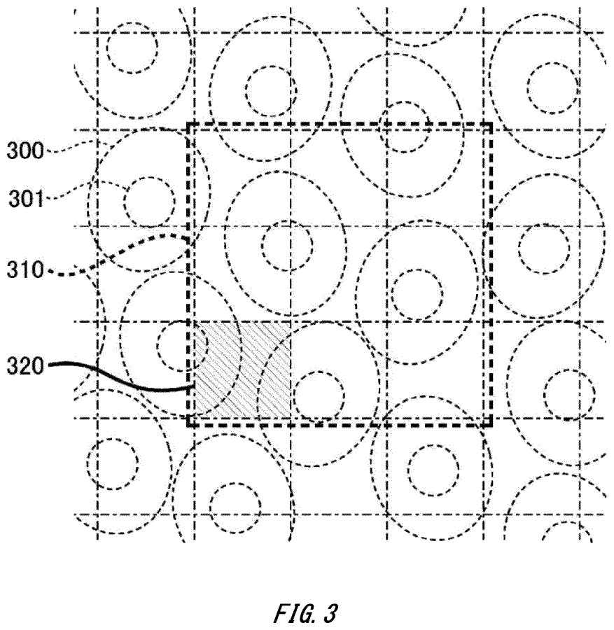

FIG. 3 is a diagram showing one example of a measuring method for all spectrums in the region of interest 310 of the sample 101. The region of interest 310 is divided into a plurality of the unit regions 320 which are even smaller regions, and each of the unit regions 320 is irradiated with excitation light to measure a light intensity of the Raman scattered light.

Herein, the unit region 320 is set in regions larger than the cell 300 included in the biological specimen, or the cell nucleus 301 of the cell 300 included in the biological specimen. More specifically, for example, the region of interest 310 with the size of 10 μm×10 μm is divided to unit regions 320, the size of which is each equal to or less than 4 μm², and the Raman spectrum is measured by irradiating each of the plurality of unit regions 320 at least once with excitation light. Thereby, the spectrums of the entire region of interest 310, that is, the all spectrums are ultimately measured.

In addition, for example, a region of interest with a rectangular shape with a size of 1 μm² is set, and 121 spectrums are measured by irradiating 121 lattice-like points formed by dividing each side with 10 division lines with excitation light having wavelength of 532 nm. The all spectrums of the region of interest 310 are also measured in this manner.

It is noted that when it is already known that a biological specimen includes a cancer cell and it is to be determined which organ the cancer derives from, that is, when an organ in which a primary focus of a biological specimen exists is determined, or when it is known that the biological specimen includes a metastatic cancer cell and the primary focus is to be determined, the identification may be at the tissue or organ level. Therefore, one region of interest may be set to be a larger region.

In addition, when the Raman spectrum is measured, after reducing the intrinsic fluorescence with photobleaching, the excitation light for measuring the Raman spectrum may be used for irradiation. Thereby, the intrinsic fluorescence of a biological specimen is significantly reduced, improving the S/N ratio of the measured spectrum. Furthermore, the shape of the regions of interest is not limited to the rectangular shape, and may be a geometrical shape such as a circle, an ellipse, and the like, as well as an irregular shape along the outline of a cell and the like.

Referring again to FIG. 2, a teacher data including information corresponding to a spectrum measured as described above is then generated (the spectrum 104). When a target for the determination by the determination device 100 is a primary focus of the cancer tissue included in a biological specimen, a teacher data including information related to the measured spectrum and the primary focus of the cancer tissue is generated. In addition, when a target for the determination by the determination device 100 is whether or not a cancer tissue exists in a biological specimen, a teacher data including information related to the measured spectrum and the presence or absence of the cancer tissue is generated. Then, the processor 171 of the determination device 100 learns the generated teacher data and generate the learned model 190 (Step S105).

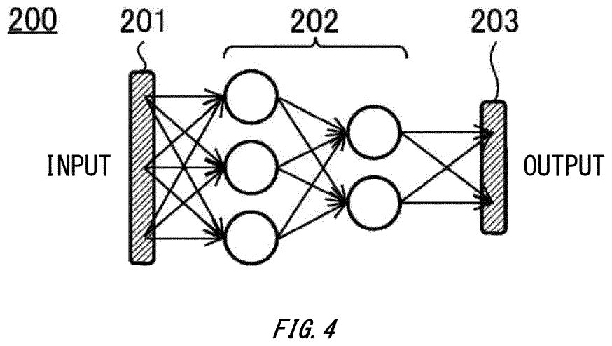

FIG. 4 is a diagram showing one example of the neural network 200 which can be used in a case where the learned model 190 stored in the storage unit 180 is generated. The neural network 200 emulating a brain neural circuit in human is formed, for example, in the processor 171 and has the input layer 201, the hidden layer 202, and the output layer 203.

The input layer 201 adjusts the weight of the activation function when an input signal is passed to the hidden layer 202. Then, after the adjustment of weight is repeated depending on the number of layers of the hidden layer 202, the signal passed to the ultimate output layer 203 is output. The output layer 203 outputs, for example, a probability that the input signal corresponds to any of the option prepared in advance.

The output probability is examined, and the adjustment of the weight is repeated until an appropriate output signal is output. Thus, the learned model 190 is generated which includes an activation function having a weight ultimately adjusted. The learned model 190 which is thus generated is stored in the storage unit 180 of the determination device 100 (Step S106). In addition, by being stored in the storage unit 180, the learned model 190 can be referred to from the processor 171. Therefore, the determination device 100 can refer to the learned model and perform a determination process.

It is noted that, for the generation of the learned model, at least one machine-learning method may be performed which is selected form the group including neural network, support vector machine, decision tree, Bayesian network, linear regression, multivariate analysis, logistic regression analysis, and determination analysis. In addition, there is no particular limit to the number of layers of the hidden layer 202.

Furthermore, the teacher data used in a case where a learned model is generated may use a representative spectrum which is processed to be suitable to a machine-learning from all spectrums. The representative spectrum is a single spectrum which represents individual regions of interest, and may be, for example, a sum of all spectrums of the regions of interest in the sample 101, or an arithmetic mean calculated by dividing the sum by the number of the spectrums.

Still further, although the determination device 100 itself is used to generate a teacher data in the above-mentioned example, another device may be used to generate a teacher data. In addition, when a plurality of the determination device 100 exists, a teacher data generated by one determination device 100 may be used in another determination device 100. Still further, the learned model 190 to be stored in the storage unit 180 may use the learned model 190 generated by one determination device 100 in a plurality of other determination device.

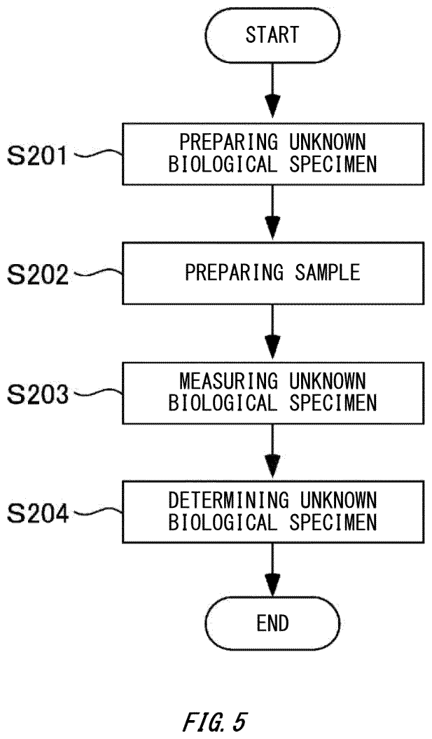

FIG. 5 is a flow diagram showing a determination procedure for a biological specimen by the determination device 100. When the determination device 100 determines an unknown biological specimen, a biological specimen which is the target for determination is first prepared (Step S201). Then, corresponding to Step S102 of a process to generate the learned model 190 shown in FIG. 2 (Step S202), after the prepared biological specimen is processed into the sample 101, the spectrum of a biological specimen is measured in a similar manner to the above-mentioned process (Step S203).

The spectrum is thus measured from the biological specimen, and the determination device 100 which generated the input data based on the spectrum of the sample 101 refers to the learned model 190 and performs the determination process to make a determination for an unknown biological specimen (Step S204). Herein, the determination by the determination device 100 is, for example, whether or not a biological specimen includes a cancer tissue, which is determined by the learned model 190 which learned the teacher data. Alternatively, the determination by the determination device 100 is, for example, a type of the primary focus of the cancer tissue included in a biological specimen, which is determined by the learned model 190 which learned the teacher data through learning.

FIG. 6 is the block diagram showing the configuration of the determination unit 210 which is formed in the determination device 100 and performs the determination process. The determination unit 210 is formed such that it includes the processor 171 which acquires the input data from the measurement unit including at least one of the front detection unit 150 and the rear detection unit 160, and the storage unit 180 which stored the learned model 190.

At least one of the front detection unit 150 and the rear detection unit 160 measures, as the measurement unit, a spectrum from a biological specimen placed on the stage 110 as the sample 101. The processor 171 performs a determination process using data which is obtained by performing a process such as removing noise components or standardizing a signal on the measured spectrum data, as the input data.

The learned model 190 stored in the storage unit 180 is referred to in a case where the processor 171 as the determination unit 210 performs the determination process. The storage unit 180 may incorporate in the processor 171, or may be a storage medium connected to the processor 171. Alternatively, the storage unit 180 may be a storage medium to which the processor 171 refers through a communication line and which is disposed outside.

In the determination unit 210, when input data measured from the biological specimen is input through the processor 171, the learned model 190 returns, to the processor 171, a determination result likely to correspond to the information. The result of the determination process by the learned model 190 is output to the user through the processor 171 as a determination result of the cancer tissue for the input data.

Then, a process is described which is performed for a spectrum measured in at least one of the front detection unit 150 and the rear detection unit 160 when the teacher data is generated, and when input data is determined based on an unknown biological specimen.

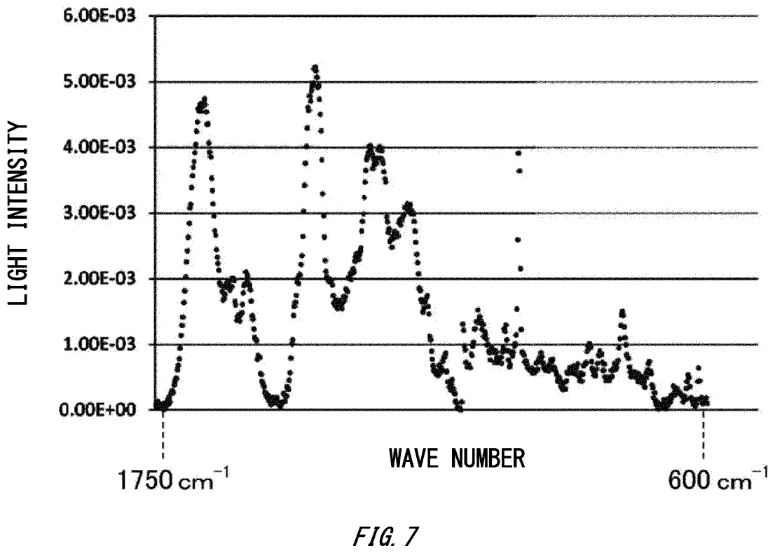

FIG. 7 is a diagram showing one example of a spectrum measured from the sample 101. When the illustrated spectrum is learned as a representative spectrum, the number of dimensions is 756. However, when this data is used for a machine-learning or a determination with a learned model, it is preferable to reduce the dimensions.

In other words, the ultimate determination accuracy can be improved by removing unnecessary data, components to which the spectrum of a biological specimen is not reflected from the above-mentioned representative spectrum, and the like, and then reducing the dimensions of spectrum data for a determination process to make the data suitable to the process such as machine-learning. When these processes are performed, a standardization process may be performed in advance so that an integrated value (an area of a region enclosed by the spectrum and the horizontal axis) of the representative spectrum becomes a predetermined value (for example 1).

As one process for the representative spectrum, a spectrum which is derived from those other than the biological specimen included in the sample 101 may be removed. In this case, those other than the biological specimen include, for example, a spectrum of reagent and the like used in a case where the sample 101 is prepared, a spectrum of glass which forms a container to support the biological specimen in the sample 101, a spectrum of an intrinsic fluorescence produced in the biological specimen, and the like.

In the above-mentioned example, it is assumed that removing the representative spectrums at both end of the measured region among the representative spectrums does not affect the determination. Thus, the dimensions of the spectrum data can be reduced by removing regions of, for example, 500-600 cm⁻¹ or 1750-1798 cm⁻¹. In this manner, the learned model 190 may be generated with the machine-learning of the spectrums in a band whose wavelength is equal to or higher than 1750 cm⁻¹ and equal to or lower than 600 cm⁻¹. Similarly, the spectrum which is the target for determination by the determination device 100 may be limited to the wavelength which is equal to or higher than 1750 cm⁻¹ and equal to or lower than 600 cm⁻¹.

One example of the spectrums which is clearly of no-biological specimen includes a spectrum of the paraffin in a preparation process of the biological specimen. With the process to remove a peak region of the paraffin from the representative spectrum, the dimensions in the learning can be reduced, while the S/N ratio of the spectrum data can be improved.

As an example, in the spectrum shown in FIG. 7, the spectrum data which has initially 756 dimensions can be reduced to 641 dimensions by removing 87 spectrums positioned at both ends of the spectrum, and 28 spectrums mentioned below corresponding to the peak region of the paraffin used to prepare the sample 101.

For 888 cm⁻¹, four spectrums positioned at 886-891 cm⁻¹

For 1061 cm⁻¹, five spectrums positioned at 1057-1064 cm⁻¹

For 1131 cm⁻¹, five spectrums positioned at 1128-1135 cm⁻¹

For 1293 cm⁻¹, nine spectrums positioned at 1287-1301 cm⁻¹

For 1366 cm⁻¹, five spectrums positioned at 1363-1370 cm⁻¹

In this manner, the ultimate determination accuracy can be improved by removing spectrums other than those of the biological specimen. In addition, by reducing the dimensions of the spectrum data which is the target for determination, the processing load of the processor 171 for the determination can be alleviated, while the processing speed can be improved.

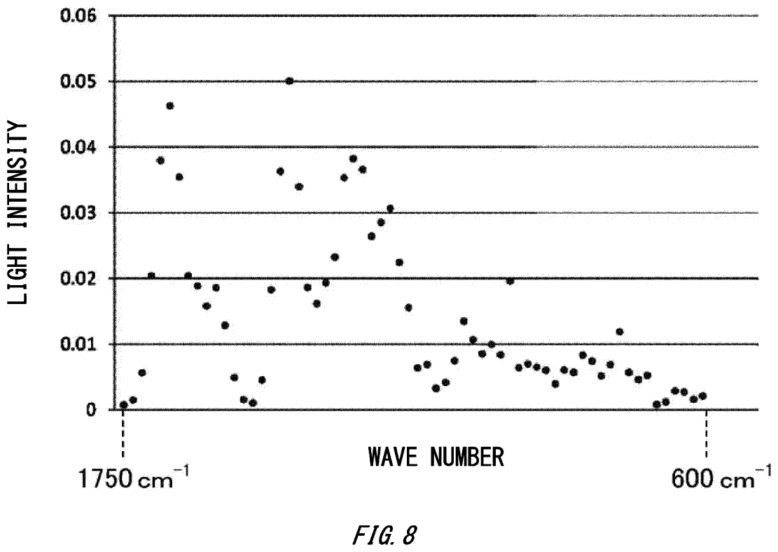

FIG. 8 shows an example in which the dimensions are reduced one tenth by averaging each 10 representative spectrums shown in FIG. 7 in the wave number direction. As illustrated, the spectrum reduced to 641 dimensions at the above-mentioned step is further reduced to 64 dimensions.

Furthermore, the representative spectrum obtained in the above-mentioned manner may include data improper for determination. Examples for improper spectrums include data in which the spectrum of the intrinsic fluorescence intensity is too large. In addition, other examples of improper spectrums include data in which the spectrum derived from the biological specimen is too small.

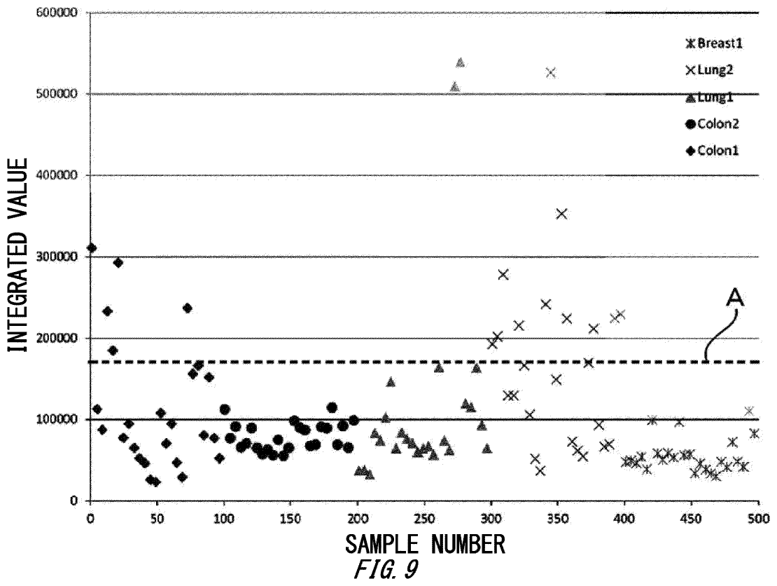

FIG. 9 is a diagram showing an example in which the spectrum of the intrinsic fluorescence intensity is too large. In the illustrated example, 125 spectrums are measured for five types of biological specimen including breast cancer, lung cancer 1, lung cancer 2, colon cancer 1, and colon cancer 2. For the region of 500-1800 cm⁻¹, the integrated value of the spectrum of the intrinsic fluorescence is obtained, the integrated value 180000 is defined as a boundary as shown as the dotted line A, and the higher representative spectrum is removed.

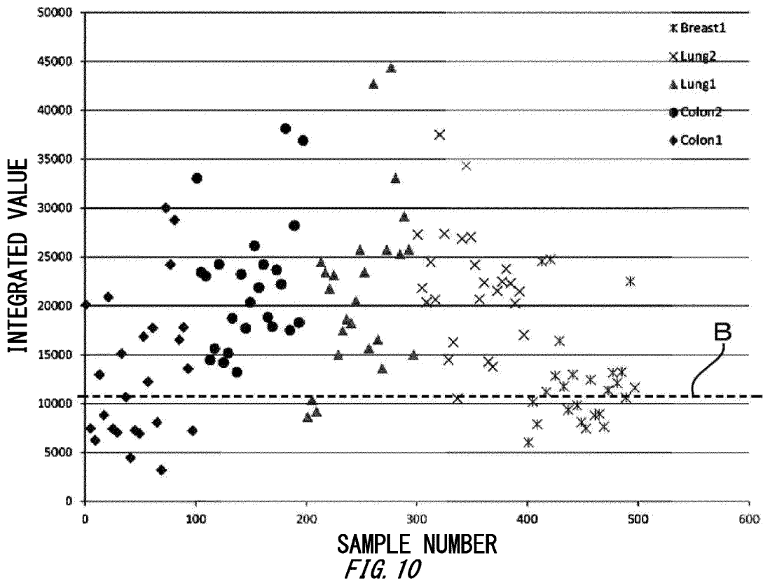

FIG. 10 is a diagram showing the example in which the spectrum of the Raman scattered light produced from the biological specimen is too small. In the illustrated example, 125 spectrums are measured for five types of biological specimen including breast cancer, lung cancer 1, lung cancer 2, colon cancer 1, and colon cancer 2. For the region of 500-1800 cm⁻¹, the integrated value of the spectrum derived from the biological specimen is obtained, the integrated value 10000 is defined as a boundary as shown as the dotted line B, and the lower representative spectrum is removed. With this process, the dimensions of the representative spectrum data can be reduced to 333.

The above-mentioned process can delete the measurement data which is difficult to separate the spectrum derived from the biological specimen and the noise and does not contribute to improve the determination accuracy. It is noted that the all spectrums may be measured again by setting another region of interest instead of the deleted spectrum.

The process for the measurement spectrum as described above is similarly performed in a case where the teacher data which is used to generate the learned model is create and in a case where the sample 101 including the biological specimen is determined. Thus, by generating a learned model using a cancer tissue known to be a cancer or a cancer tissue whose primary focus is known, and storing it in the storage unit 180 of the determination device 100, when the sample 101 including the biological specimen is provided for the determination, the determination device 100 determines whether or not the biological specimen included in the sample 101 is cancer, or the primary focus of the cancer tissue.

### EXPERIMENTAL EXAMPLE 1

As shown in FIG. 8, by using the spectrum data whose dimensions are reduced by averaging each 10 spectrums, and the spectrum data whose dimensions are still not reduced, the determination result of the biological specimen whose primary focus is the colon cancer is compared for the two biological specimens. As described below, it is shown that the change in the determination rate due to the reduction of dimensions is small. It is noted that the determination rate indicates the ratio of the number of correct determination result to the number of all determinations.

Biological specimen 1;

Before averaging: 84.5%

(Peaks: 1665 cm⁻¹, 1406 cm⁻¹, 1581 cm⁻¹, 1004 cm⁻¹)

After averaging: 85.5%

(Peaks: 1350 cm⁻¹, 1614 cm⁻¹, 1367 cm⁻¹, 1598 cm⁻¹)

Biological specimen 2;

Before averaging: 94%

(Peaks: 1036 cm⁻¹, 1282 cm⁻¹, 1430 cm⁻¹, 878 cm⁻¹)

After averaging: 95%

(Peaks: 1450 cm⁻¹, 1266 cm⁻¹, 721 cm⁻¹, 1434 cm⁻¹)

It is noted that the above-mentioned “Peaks” indicates the value of intensity at a position of each spectrum. In the above-mentioned example, two intensity ratios are made by using four spectrum intensities of 1665 cm⁻¹, 1406 cm⁻¹, 1581 cm⁻¹, 1004 cm⁻¹, and a linear discriminant analysis is performed by creating a two-dimensional scatter plot.

### EXPERIMENTAL EXAMPLE 2

For the sample 101 known to include five types of biological specimen including breast cancer, lung cancer 1, lung cancer 2, colon cancer 1, colon cancer 2, 125 representative spectrums of the regions of interest are obtained in total. The size of the individual region of interest is 10 μm×10 μm. In the regions of interest, 121 spots with the interval of 1 μm are irradiated with excitation light for five seconds, and 121 spectrums are obtained for each region of interest. Furthermore, for each region of interest, the sum of the spectrum is divided by 121 to obtain the representative spectrum of the regions of interest. Thus, as shown in FIGS. 9 and 10, 125 representative spectrums are obtained.

Furthermore, after standardizing so that the integrated value of each representative spectrum is 1, by removing the regions of 500-600 cm⁻¹ and 1750-1798 cm⁻¹ positioned at both ends of the spectrum and the peak region of the paraffin, the dimensions of the spectrum data is reduced from 756 dimensions to 641 dimensions. Furthermore, the dimensions are reduced to 64 dimensions by averaging each 10 data.

Then, as shown in FIG. 9, for each of 125 representative spectrums, the integrated value for the region of 500-1800 cm⁻¹ of the intrinsic fluorescence spectrum is obtained and the representative spectrum equal to or higher than 180000 is removed. Furthermore, as shown in FIG. 10, the integrated value for the region of 500-1800 cm⁻¹ of the spectrum derived from the biological specimen is obtained and the representative spectrum equal to or lower than 10000 is removed. With these processes, the representative spectrum is reduced to 333.

Among these 333 representative spectrums, 283 representative spectrums are learned by the neural network shown in FIG. 4 as teacher data including information about a primary focus, and the learned model 190 is generated. The learned model 190 which is generated is stored in the storage unit 180, and the determination device 100 is enabled to perform the determination for the other 50 representative spectrum. As a result, the primary focus determination rate was 92%.

### EXPERIMENTAL EXAMPLE 3

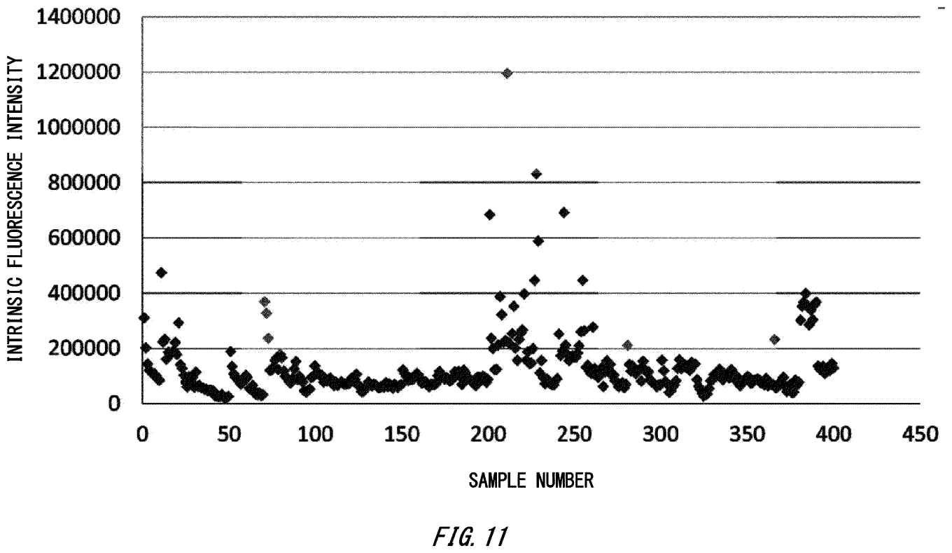

By using a biological specimen including a colon cancer, and, among slices cut out to create the biological specimen, a biological specimen of the normal tissue adjacent to the region including a cancer cell, the training data and the test data are prepared in a procedure similar to experimental example 2. Corresponding to the case shown in FIG. 9, FIG. 11 is a diagram showing a result of obtaining the integrated value for the region of 500-1800 cm⁻¹ of the spectrum of the intrinsic fluorescence for each of 500 representative spectrums. The representative spectrum whose integrated value is equal to or higher than 180000 is removed from the illustrated data. Furthermore, corresponding to the case shown in FIG. 10, the integrated value for the region of 500-1800 cm⁻¹ of the spectrum derived from the biological specimen is obtained and the representative spectrum equal to or lower than 10000 is removed.

The result of determining 50 spectrums as test data among the data which is thus reduced shows that the determination rate is 90.2%. In this manner, the determination device 100 can also determine whether or not the biological specimen includes a cancer tissue.

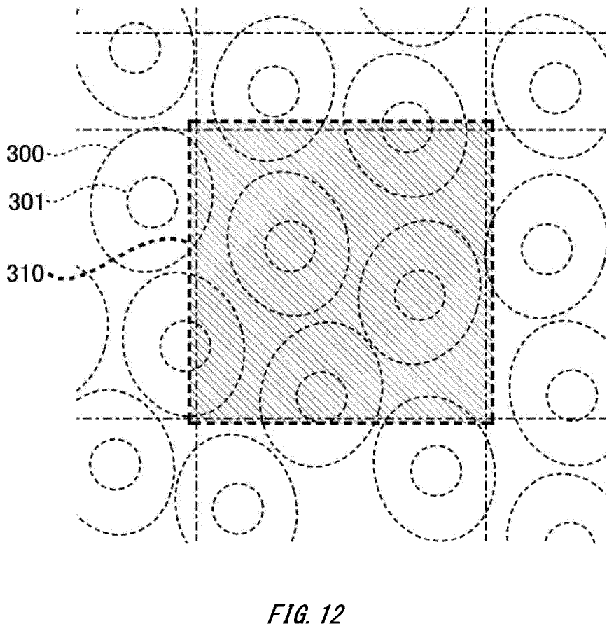

FIG. 12 is a diagram to exemplify another method to measure the all spectrums from the sample 101. This method is a method to measure the Raman spectrum by irradiating the entire region of the regions of interest with the excitation light with uniform intensity. That the irradiated regions are uniformly distributed means that the irradiated regions are distributed substantially without bias. Whether or not it is uniformly distributed can be confirmed with a well-known method which evaluates the uniformity of distribution.

For example, it refers to the case in which, when the regions of interest is divided into n regions with equal areas (n is any integer equal to or more than 2), the number or area of the irradiated regions included in each divided region is substantially equal. For example, the number of all spectrums which is acquired may be, for each region of interest, equal to or more than 50, 60, 70, 80, 90, or 100, depending on the area of the regions of interest. In addition, the measurement result may be obtained as a total of the all measurement result in the region of interest. Furthermore, the average spectrum obtained by dividing the total by the number of irradiation may be used.

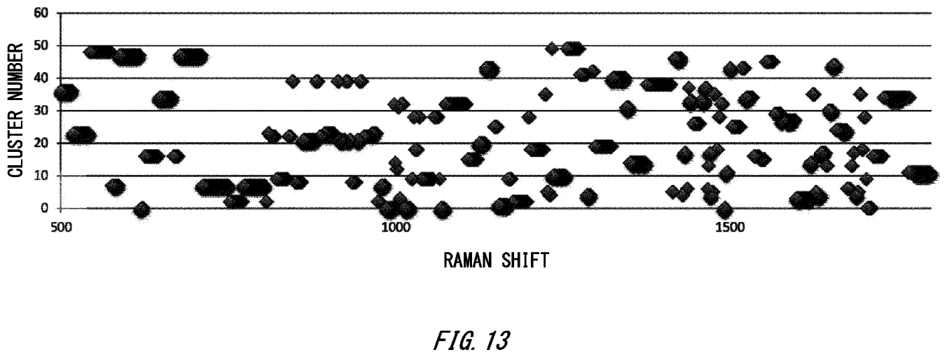

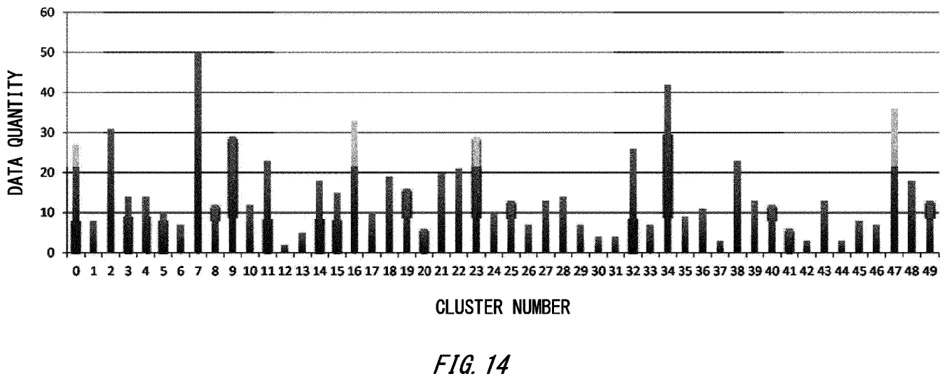

In addition, as a method to reduce the dimensions of the spectrum data, another well-known method such as clustering can be used. FIG. 13 and FIG. 14 indicate the reduction in the dimensions of the representative spectrum data with the clustering. FIG. 13 shows 756 peaks in the representative spectrum. By classifying this to 50 clusters and replacing each cluster with one peak, the spectrum can be reduced to 50 dimensions as shown in FIG. 14. The clustering can reduce more spectrums than a simple averaging while reflecting the property of the biological specimen, since it averages the peaks, as a cluster, with the same characteristics.

While the embodiments of the present invention have been described, the technical scope of the invention is not limited to the above described embodiments. It is apparent to persons skilled in the art that various alterations and improvements can be added to the above-described embodiments. It is also apparent from the scope of the claims that the embodiments added with such alterations or improvements can be included in the technical scope of the invention.

The operations, procedures, steps, and stages of each process performed by an device, system, program, and method shown in the claims, embodiments, or diagrams can be performed in any order as long as the order is not indicated by “prior to,” “before,” or the like and as long as the output from a previous process is not used in a later process. Even if the process flow is described using phrases such as “first” or “next” in the claims, embodiments, or diagrams, it does not necessarily mean that the process must be performed in this order.

### EXPLANATION OF REFERENCES

100 determination device, 101 sample, 110 stage, 111 stage scanner, 120 objective optical system, 121 front objective lens, 122 rear objective lens, 130 light source device, 131, 132 light source, 139 combiner, 140 irradiation optical system, 141 galvano scanner, 142 scan lens, 143 primary image plane, 144 reflection mirror, 145 collimator lens, 150 front detection unit, 151 dichroic mirror, 152, 153, 162, 163 relay lens, 154, 164 band pass filter, 155, 165 spectrometer, 160 rear detection unit, 161 reflection mirror, 170 control unit, 171 processor, 172 mouse, 173 keyboard, 174 display unit, 180 storage unit, 190 learned model, 200 neural network, 201 input layer, 202 hidden layer, 203 output layer, 210 determination unit, 300 cell, 301 cell nucleus, 310 region of interest, 320 unit region

What is claimed is:

1. A determination device, comprising a determination unit including a processor and a storage unit, wherein
the processor acquires input data based on an optical spectrum obtained by measuring a biological specimen,
the storage unit stores a learned model which is generated by learning teacher data including an optical spectrum obtained by measuring a cancer tissue whose primary focus is known, and
the determination unit determines a primary focus of the biological specimen from the input data by using the learned model.

2. The determination device according to claim 1, wherein
the storage unit stores a learned model obtained by learning, as teacher data, a first optical spectrum of a first cancer cell derived from a first primary focus and a second optical spectrum of a second cancer cell derived from a second primary focus which is different from the first primary focus, and
the determination unit determines whether a primary focus of the biological specimen is the first primary focus or the second primary focus.

3. The determination device according to claim 1, wherein
the storage unit stores a learned model obtained by learning, as teacher data, an optical spectrum of a colon cancer, an optical spectrum of a breast cancer, and an optical spectrum of a lung cancer, and
the determination unit determines whether or not a primary focus of the biological specimen is any of a colon cancer, a breast cancer, and a lung cancer.

4. The determination device according to claim 1, wherein the storage unit further stores a learned model obtained by learning, as teacher data, an optical spectrum measured from a normal tissue.

5. The determination device according to claim 1, wherein the learned model is generated by means of machine-learning of optical spectrums in a band whose wavelength is equal to or higher than 1750 cm⁻¹ and equal to or lower than 600 cm⁻¹.

6. The determination device according to claim 1, wherein the learned model includes information corresponding to a total sum of optical spectrums, in a region of interest, measured by irradiating each of a plurality of unit regions which is a part of the region of interest at least once with an excitation light, wherein the region of interest is a target for measurement for an optical spectrum.

7. The determination device according to claim 1, wherein the learned model includes information corresponding to an arithmetic mean of optical spectrums, in a region of interest, measured by irradiating each of a plurality of unit regions which is a part of the region of interest at least once with an excitation light, wherein the region of interest is a target for a measurement for an optical spectrum.

8. The determination device according to claim 1, wherein the learned model includes information corresponding to a sum of all spectrums obtained by irradiating, with an excitation light which is uniformly distributed, an entire region of interest which is a target for a measurement for an optical spectrum.

9. The determination device according to claim 1, wherein the learned model includes information corresponding to an average spectrum which is obtained by dividing a sum of all spectrums by a number of optical spectrums, wherein the all spectrums is obtained by irradiating, with an excitation light which is distributed uniformly, an entire region of interest which is a target for a measurement for an optical spectrum.

10. The determination device according to claim 1, wherein the learned model is generated by means of machine-learning of data of the optical spectrum whose dimensions are reduced.

11. The determination device according to claim 1, where the learned model is generated by means of machine-learning using at least one of following methods: neural network, support vector machine, decision tree, Bayesian network, linear regression, multivariate analysis, logistic regression analysis, and determination analysis.

12. The determination device according to claim 1, wherein the determination unit refers to, as the learned model, an optical spectrum obtained by removing an optical spectrum in which an integrated value of an intrinsic fluorescence spectrum is higher than a predetermined upper limit value, and an optical spectrum in which an integrated value of intrinsic fluorescence spectrum is lower than a predetermined lower limit value.

13. The determination device according to claim 1, wherein the determination device includes a measurement unit to detect a Raman scattered light derived from a biological specimen.

14. A determination method, comprising:
acquiring input data based on an optical spectrum obtained by measuring a biological specimen,
referring a learned model generated by learning teacher data including an optical spectrum obtained by measuring a cancer tissue whose primary focus is known, and
determining a primary focus of the biological specimen from the input data by using the learned model.

15. A determination program which causes a computer to perform a step of:
acquiring input data based on an optical spectrum obtained by measuring a biological specimen,
referring a learned model generated by learning teacher data including an optical spectrum obtained by measuring a cancer tissue whose primary focus is known, and
determining a primary focus of the biological specimen from the input data by referring the learned model.
